(19) United States
(12) Patent Application Publication
(10) Pub. No.: US 2022/0310234 A1
(43) Pub. Date: Sep. 29, 2022

(54) SYSTEM AND METHOD FOR PROVIDING PERSONALIZED HEALTH FUNCTIONAL FOOD INFORMATION

(71) Applicant: LOGSYNK CO., LTD., Seoul (KR)

(72) Inventor: Soo Il LEE, Cheonan-si, Chungcheongnam-do (KR)

(73) Assignee: LOGSYNK CO., LTD., Seoul (KR)

(21) Appl. No.: 17/414,325

(22) PCT Filed: Dec. 17, 2020
(86) PCT No.: PCT/KR2020/018528

§ 371 Date: Jun. 16, 2021

(30) Foreign Application Priority Data
Aug. 6, 2020 (KR) 10-2020-0098406

(51) Int. Cl. G16H 20/60; G16H 10/20; G09B 19/00

(52) U.S. Cl. CPC G16H 20/60; G16H 10/20; G09B 19/0092

(57) ABSTRACT

The present invention is characterized by comprehensively analyzing client's genetic percentage data, client's health related information data by self-determination, and health examination information data; summing up self-phenomenon values, which are obtained by summing up the self-determination information data and health examination data and converting into percentages, and the genetic percentage data, wherein a greater health functional food recommendation weighting ratio is given to the self-phenomenon data percentage data than to the genetic percentage data for analysis; and recommending the health index item for the result value having the lowest percentage data among the analyzed result values, to provide personalized health functional food that helps to promote health.

### TECHNICAL FIELD

The present invention relates to a system and method for providing personalized health functional food information. More specifically, the present invention relates to a mobile-based system and method for providing personalized health functional food information capable of scientifically recommending the most suitable health functional food for client's health disorder through analysis of individual's health conditions and health related genes.

### BACKGROUND ART

We hope to live a long and healthy life. So, many people look for health functional foods, vitamins, restoratives, etc. that are good for health. Our ancestors considered meals as restoratives. The most important thing for promoting health is the contents of three meals a day. There are a lot of foods that can be easily found around us and help us stay healthy. It is no exaggeration to say that food refers to our body and mind. The food industry that always needs to launch novel and differentiated products catering to modem people's various desires and tastes becomes interested in healthy foodstuffs as “wellbeing” is recognized as a significant lifestyle to consumers.

It is well known that responding to health disorders in a human body, by allowing the consumers to ingest health functional foods that are produced and processed using nutrients or functional substances useful for health promotion or health care of the human body, will be a great help for health promotion of the human body.

Health functional foods refer to foods that are good for health, which are 61 items including 4 types of terpenoids, 3 types of phenols, 8 types of fatty acids and lipids, 8 types of sugars and carbohydrates, 2 types of microzymes, 1 type of amino acid and protein, 7 types of general raw materials, etc., as well as 14 types of vitamins such as vitamin A, etc., 11 types of minerals such as calcium, etc., dietary fiber, protein, and essential fatty acid. It is general to refer to foods having colors, such as tomato, spinach, red wine, nuts, broccoli, oats, salmon, garlic, green tea, blueberry, etc. Since it is difficult to ingest a broad variety of these health functional foods, protein powders serving as dietetic meals, fresh products serving as breakfast, shakes serving as meals, etc., in place of the health functional foods, are widely known.

It is very difficult for many people to select and ingest health functional foods helpful for their health or health functional foods in place of meals. When the people desire to receive a recommendation on personalized health functional foods that help to promote their health, it is necessary to receive help from experts such as doctors or nutritionists, etc. Therefore, it is required to provide information based on scientific and objective data, from which health functional foods can be easily and conveniently recommended according to a signal of health disorder.

In the past, personalized health functional food was recommended to individuals by specific experts such as nutritionists, etc. based only on simple information in consideration of simple measured numerical values such as individual's physical characteristics, diseases, etc., which lack scientific grounds.

To this end, the applicant filed the application earlier and had it granted as Korean Patent No. 10-2063119, to provide personalized health functional food information.

According to this, personalized health functional food is provided by comparing and analyzing each subdivided item of genetic percentage data, percentage data of a current skin condition by a skin measurement terminal, and percentage data of self-determination items of a subject, determining a two-digit English determination code of the current skin condition of the subject, generating a three-digit English determination code which combines the determined code with a one-digit English determination code which helps skin metabolism, and recommending personalized health functional food capable of solving skin problems according to the generated three-digit English code, thereby increasing satisfaction and reliability of the consumers.

However, the prior art technology provides health functional food based on individual's skin condition, and thus cannot provide more exact and reliable personalized health functional food corresponding to each individual's health condition.

### DETAILED DESCRIPTION OF INVENTION

Technical Task

It is an object of the present invention to recommend personalized health functional food by a comparative analysis between health related genetic testing result data, from which each individual's health condition can be scientifically detected, data obtained from phenomenological health related self-determination and an Internet of Things (IoT) measurement information terminal, and health examination information data.

Method for Solving the Technical Task

The present invention is characterized by comprehensively analyzing client's genetic percentage data, client's health related information data by self-determination, and health examination information data; summing up self-phenomenon values, which are obtained by summing up the self-determination information data and health examination data and converting into percentages, and the genetic percentage data, wherein a greater health functional food recommendation weighting ratio is given to the self-phenomenon data percentage data than to the genetic percentage data for analysis; and recommending the health index item for the result value having the lowest percentage data among the analyzed result values, to provide personalized health functional food that helps to promote health.

Also, the present invention is characterized by, if there is client's health related information data obtained by an IoT measurement information terminal, substituting the corresponding information data for self-determination information data to obtain self-phenomenon values; summing up the obtained self-phenomenon values and genetic percentage data, wherein a greater health functional food recommendation weighting ratio is given to the self-phenomenon data percentage data than to the genetic percentage data for analysis; and if the percentage data is 45% or less as a result of analysis, recommending personalized health functional food related to obesity and/or hair loss.

### Effect of the Invention

The present invention has the effects of recommending more scientific and reliable health functional food by recommending health functional food based on percentages obtained by summing up self-phenomenon values according to client's health disorder, obtained from data by health related self-determination and self-phenomenon, and health disorder information of client's genetic information data, wherein a greater health functional food recommendation weighting ratio is given to the self-phenomenon values, and of quickly and conveniently providing health functional food information data by providing the recommended health functional food items to personal smartphone or user's PC.

Also, the present invention has the effects of increasing the degree of understanding on food, preventing damages and supporting a healthy diet by analyzing health condition and recommending more specific analysis information and personalized food information with a smartphone once personal health condition information is input to the smartphone or user's PC, and of using the recommended health functional food information anytime, anywhere since the user can retrieve and confirm the information on his own using his smartphone.

### BRIEF DESCRIPTION OF DRAWINGS

FIG. 1 is a block diagram of the whole system circuit according to the present invention;

FIG. 2 is a classification table of a health related self-determination list of 37 questionnaire items related to metabolism, obesity, and hair according to the present invention;

FIG. 3 is a classification table of 21 genes related to metabolism, obesity, and hair genes according to the present invention;

FIG. 4 is a drawing illustrating an example of marked items corresponding to health disorders as a result of genetic testing for the items of metabolism, obesity regulation, and hair loss in the classification table of 21 genes related to metabolism, obesity, and hair genes according to the present invention;

FIG. 5 is a classification table of a health examination list of 8 items including immunity inflammation, cardiovascular function, hyperlipidemia, thyroid, liver function, kidney, hematologic abnormality, and diabetes, received from a health examination (blood) analysis company, for explaining the present invention;

FIG. 6 is a comparison table of a health examination list and genetic items which correspond to metabolism, for explaining the present invention; and

FIG. 7 and FIG. 7a to FIG. 7g are flowcharts for explaining a method according to the present invention.

### BEST MODE FOR CARRYING OUT THE INVENTION

The present invention relates to a mobile-based system and method for providing personalized health functional food information, capable of scientifically recommending health functional food and/or health supplement food as meal substitutes, meal replacements (powders) promoting health function, or general food promoting health function (hereinafter, health functional food), which is most suitable for a client's health disorder, through analysis of individual's health conditions and health related genes. The present invention is described in detail with reference to the accompanying drawings.

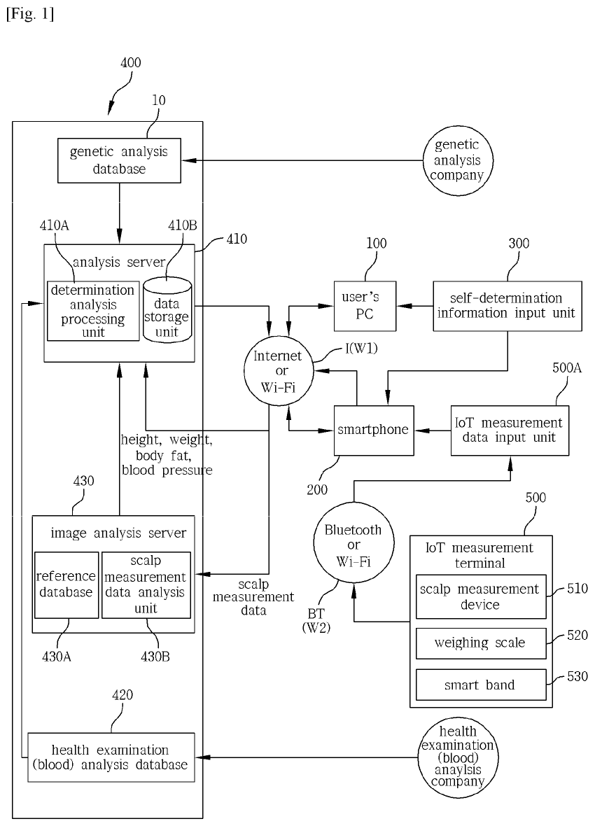

FIG. 1 is a block diagram of the whole system circuit according to the present invention.

According to the drawing, the system is configured to comprise a genetic analysis database (10) for analyzing percentage data on health related genetic testing items related to metabolism, obesity, and hair (hair loss) genes (for example, 21 genetic items), received from a genetic analysis company; a self-determination information input unit (300) for inputting, through Internet (I) or Wi-Fi (W1), self-determination percentage data obtained by checking, at a user's PC (100) and smartphone (200), each of questionnaire items of metabolism, obesity, and hair (hair loss), etc. in a health related self-determination list (for example, 37 questionnaire subdivided items) and converting into percentages using the Likert scale of measurement; a data storage unit (410B) for storing the genetic percentage data of the genetic analysis database (10) and the self-determination percentage data from the self-determination information input unit (300) and a determination analysis processing unit (410A) for comparing and analyzing input data, which constitute an analysis server (410) in a cloud server (400); a health examination (blood) analysis database (420) for analyzing and storing percentage data on health examination items (for example, 8 health examination items) received from a health examination (blood) analysis company and transmitting the data to the determination analysis processing unit (410A); and an IoT measurement data input unit (500A) for inputting, through Bluetooth (BT) or Wi-Fi (W2), percentage data of hair loss related items obtained by a scalp measurement device (510) of an IoT measurement terminal (500), percentage data of body mass index (BMI) obtained by a weighing scale (520) and a smart band (530), and percentage data of metabolic regulation obtained from blood pressure measurement, wherein if there is data input by the IoT measurement data input unit (500A), the percentage data of body mass index obtained by the weighing scale (520) and smart band (530) and the percentage data of metabolic regulation obtained from blood pressure measurement, instead of the corresponding items of the self-determination percentage data obtained by checking each of the questionnaire items in the self-determination list and converting into percentages using the Likert scale of measurement, are input to the analysis server (410) for analysis, the percentage data of hair loss related items obtained by the scalp measurement device (510) is input to an image analysis server (430) which comprises a reference database (430A) and a scalp measurement data analysis unit (430B) and analyzed by the analysis server (410) to analyze client's genetic percentage data, client's body related information data by self-determination, and health examination information data, metabolism related health functional food recommendation determination values, which are calculated by giving a health functional food recommendation percentage weighting ratio 50%:50% (by multiplication) to each of metabolism related health functional food recommendation self-phenomenon values, calculated as average percentage data from the questionnaire items in the self-determination list, and metabolism related health functional food recommendation health examination determination values, calculated as average percentage data from health examination, and summing up the values, and respective metabolism related health functional food genetic determination values are summed up, a health functional food recommendation weighting ratio 80%:20% (by multiplication) is given to each of the metabolism related health functional food recommendation determination values and the metabolism related health functional food genetic determination values, to calculate health functional food recommendation result values for metabolism improvement, health functional food recommendation determination codes for metabolism improvement are decided, which correspond to the respective calculated health functional food recommendation result values, and a health functional food recommendation determination code of a metabolism item which is calculated as a health functional food recommendation result value having the lowest percentage data among the health functional food recommendation result values is recommended for health functional food.

In the above, metabolism in the metabolism related health functional food recommendation determination values, each of the health functional food recommendation result values for metabolism improvement, and the health functional food recommendation determination codes for metabolism improvement are items for improvement of blood circulation, cell activation, antioxidation, metabolic regulation and/or obesity.

Also, the present invention can recommend personalized health functional food for improvement of obesity and hair loss in case the percentage data of each of the health functional food recommendation result values for improvement of obesity, body mass index, and hair loss calculated by giving a health functional food weighting ratio 80%:20% to each of the health functional food recommendation self-phenomenon values related to obesity, body mass index, and hair loss and the health functional food genetic determination values related to obesity, body mass index, and hair loss, is 45% or less.

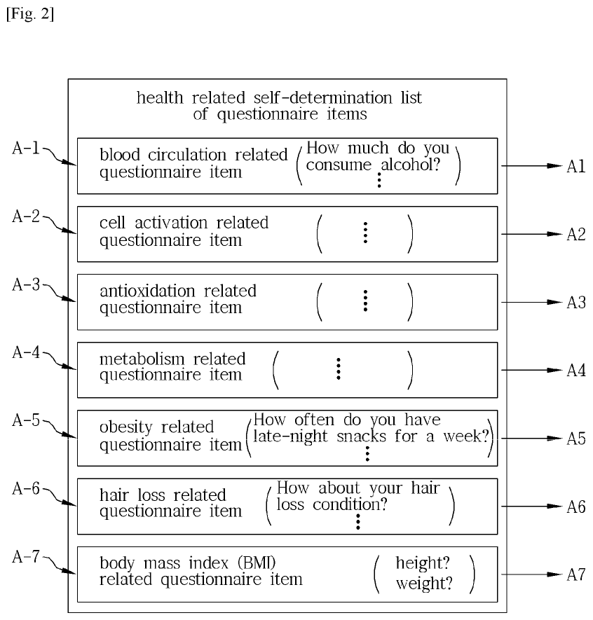

FIG. 2 is a classification table of a health related self-determination list of questionnaire items related to metabolism and/or obesity and hair, etc., according to the present invention, which is a drawing illustrating an example of part of list of questionnaire items.

According to the drawing, each of the questionnaire items in the health related self-determination list related to metabolism, obesity, and hair, etc., is checked and measured using the Likert scale of measurement through the self-determination information input unit (300), and the measured values are input to the determination analysis processing unit (410A) of the analysis server (410) through Internet (I) or Wi-Fi (W1) to calculate self-determination percentage data.

That is, average percentage data obtained by summing up percentage data of a plurality of questionnaire items for each of blood circulation related questionnaire item (A-1), cell activation related questionnaire item (A-2), antioxidation related questionnaire item (A-3), metabolism related questionnaire item (A-4), obesity related questionnaire item (A-5), hair loss related questionnaire item (A-6), and body mass index related questionnaire item (A-7), among the self-determination percentage data converted into percentages using the Likert scale of measurement, is calculated as health functional food recommendation self-phenomenon values (A1), (A2), (A3), (A4), (A5), (A6), and (A7) at the determination analysis processing unit (410A) of the analysis server (410). The health functional food recommendation self-phenomenon values (A1), (A2), (A3), (A4), (A5), (A6), and (A7) are self-phenomenon values obtained by which the client himself checks health condition questionnaire items.

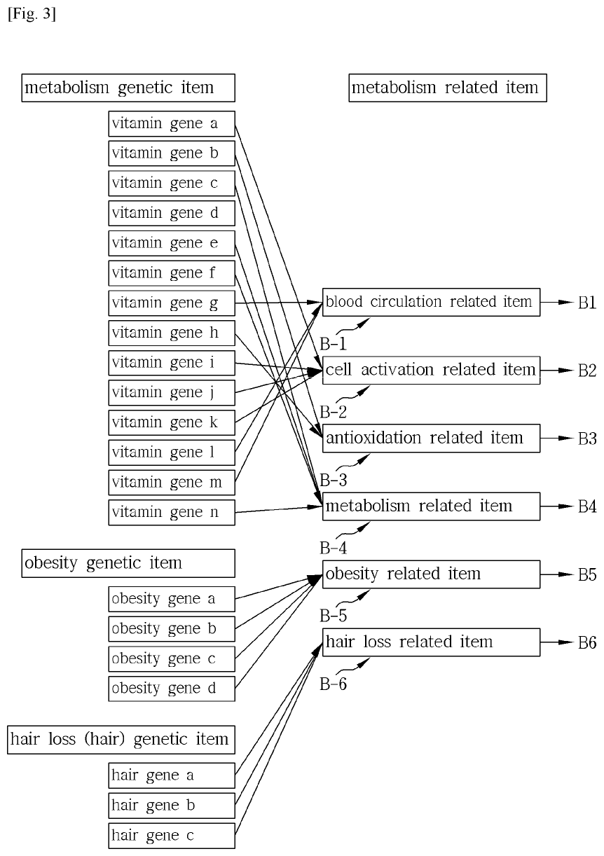

FIG. 3 is a classification table of genes related to metabolism, obesity, and hair genes according to the present invention.

According to the drawing, genes are classified into vitamin gene a˜vitamin gene n as metabolism genetic items, obesity gene a˜obesity gene d as obesity genetic items, and hair gene a˜hair gene c as hair (hair loss) genetic items. Average percentage data obtained by summing up percentage data on genetic testing items for each of metabolism related blood circulation related item (B-1), cell activation related item (B-2), antioxidation related item (B-3), metabolism related item (B-4), obesity related item (B-5), hair loss related item (B-6), and body mass index related item (B-7), among the metabolism genetic items, is calculated as health functional food recommendation genetic determination values (B1), (B2), (B3), (B4), (B5), (B6), and (B7) as a result of genetic testing at the determination analysis processing unit (410A) of the analysis server (410).

For example, as to the blood circulation improvement item (B-1), percentage data of each of vitamin gene g, vitamin gene 1, and vitamin gene m, which correspond to blood circulation improvement metabolism items, is summed up, and then the average percentage data for this item may be set as a health functional food recommendation genetic determination value (B1) for blood circulation improvement.

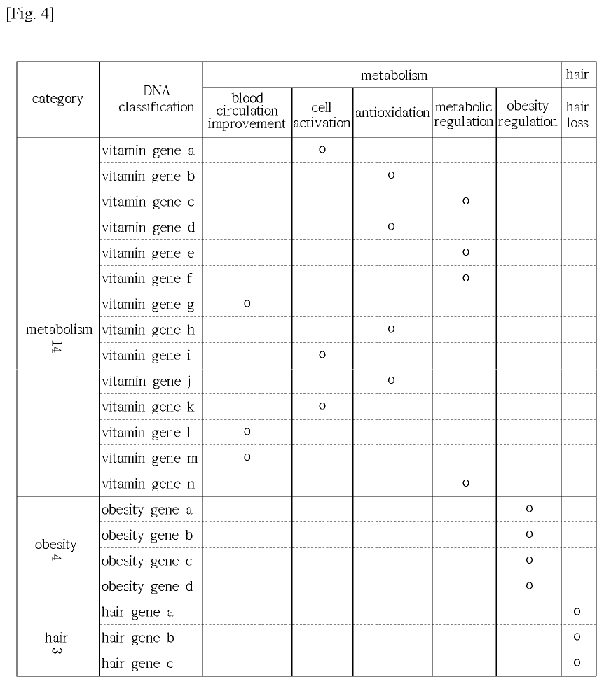

FIG. 4 is a drawing illustrating an example of marked items corresponding to health disorders as a result of genetic testing for each of the items of metabolism, obesity regulation, and hair loss in the classification table of 21 genes related to metabolism, obesity, and hair genes according to the present invention.

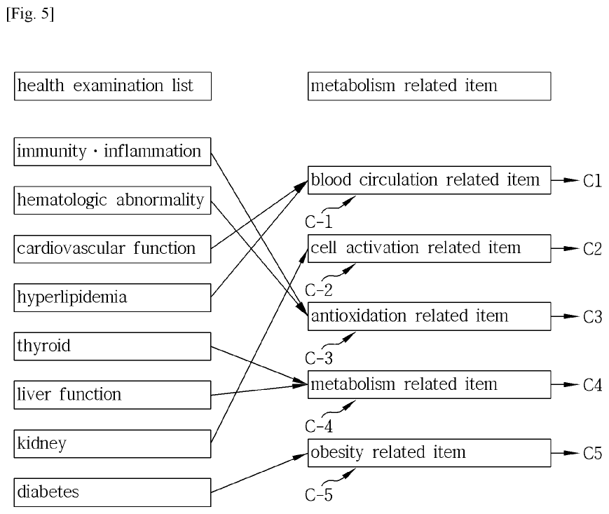

FIG. 5 is a classification table of a health examination list including immunity inflammation, cardiovascular function, hyperlipidemia, thyroid, liver function, kidney, hematologic abnormality, and diabetes, etc., received from a health examination (blood) analysis company. The drawing illustrates an example in which percentage data obtained by summing up health examination item percentages for each of the metabolism related items including blood circulation related item (C-1), cell activation related item (C-2), antioxidation related item (C-3), metabolism related item (C-4), obesity related item (C-5), etc., which correspond to the health examination list, is calculated as health functional food recommendation health examination determination values (C1), (C2), (C3), (C4), and (C5) at the determination analysis processing unit (410A) of the analysis server (410).

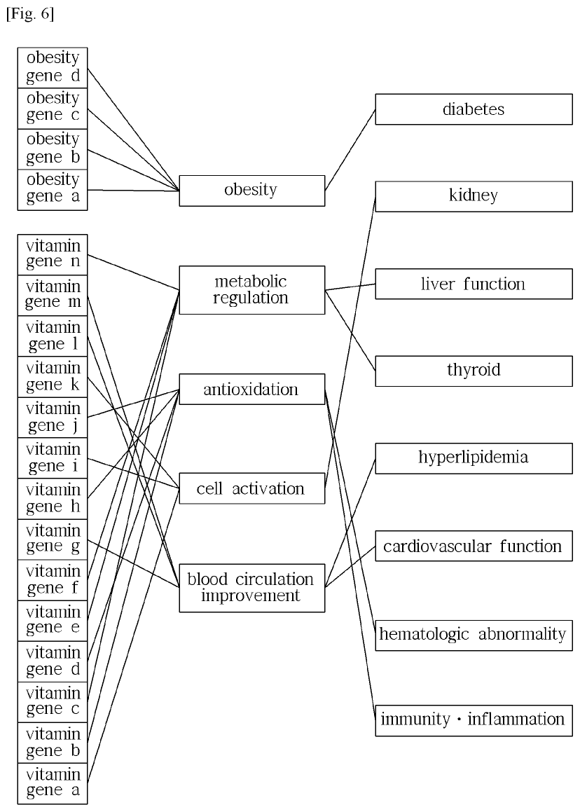

FIG. 6 is a comparison table of a health examination list and genetic items which correspond to metabolism items, for explaining the present invention. The present invention is explained in detail as in the following examples.

A. Hereinafter, in case there is percentage data of health examination (blood) analysis, a method for recommending health functional food corresponding to metabolism items by a comparison between self-phenomenon values, health examination determination values, and genetic determination values, is described.

a) Method for recommending health functional food corresponding to metabolism items

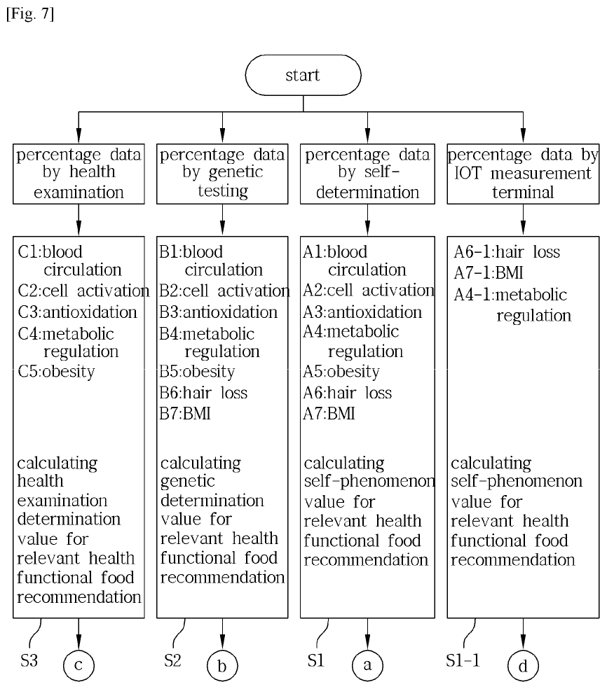

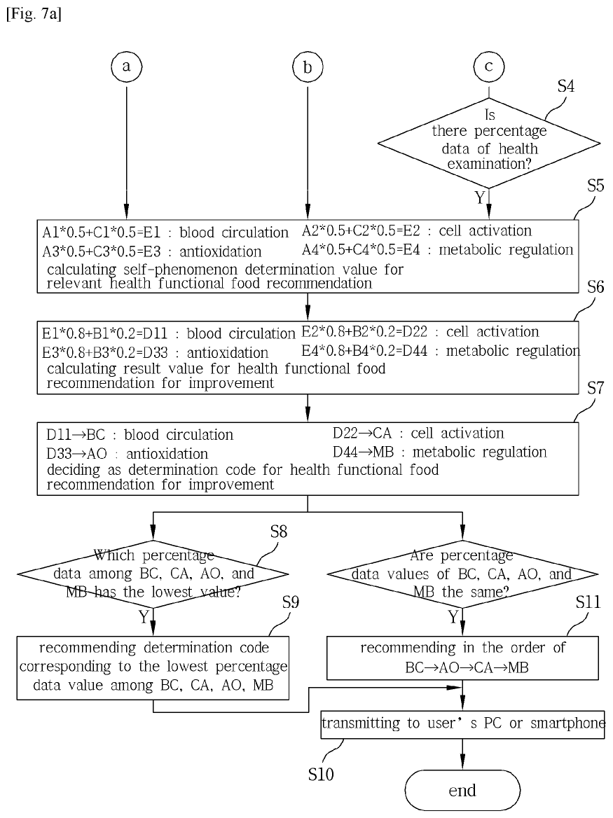

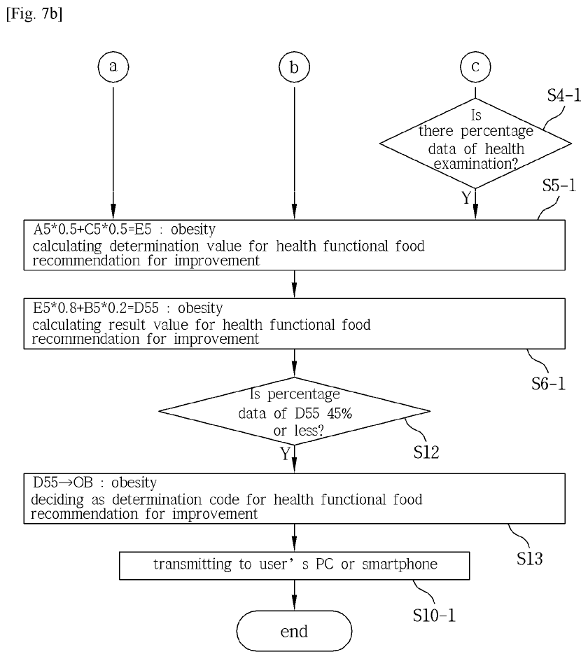

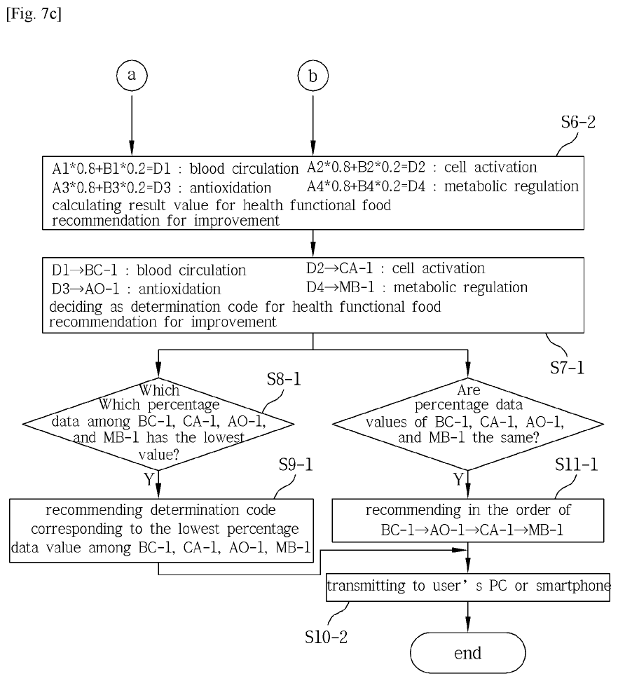

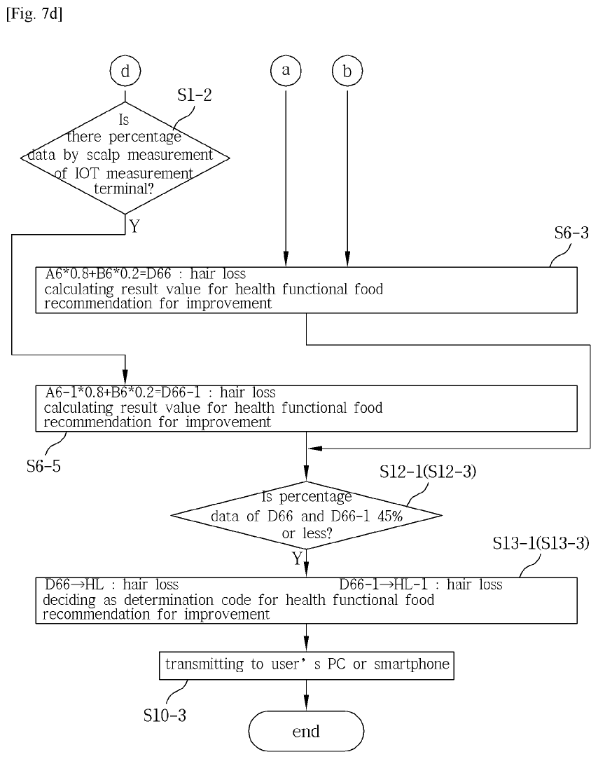

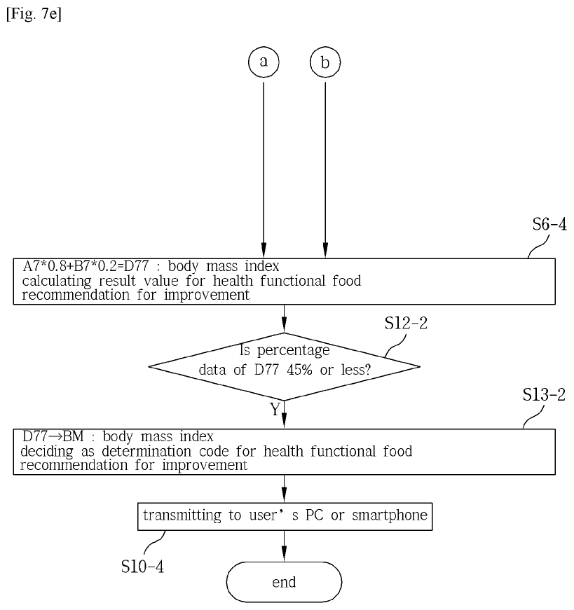

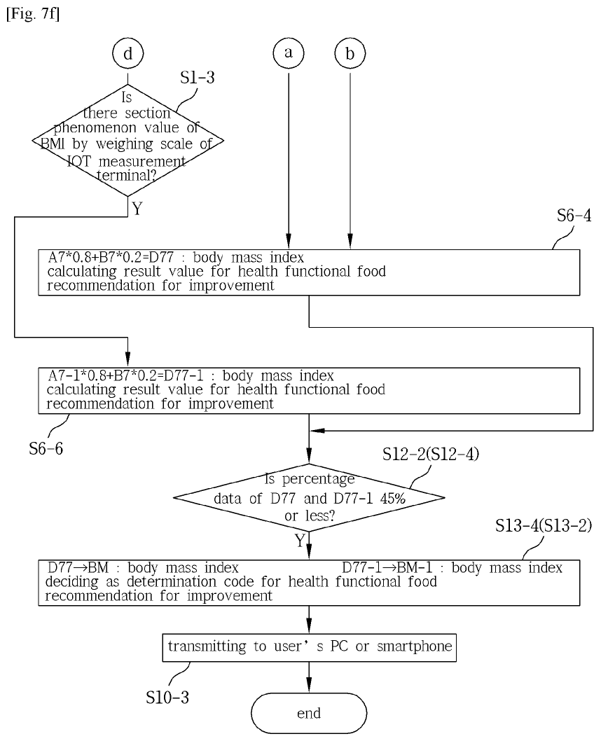

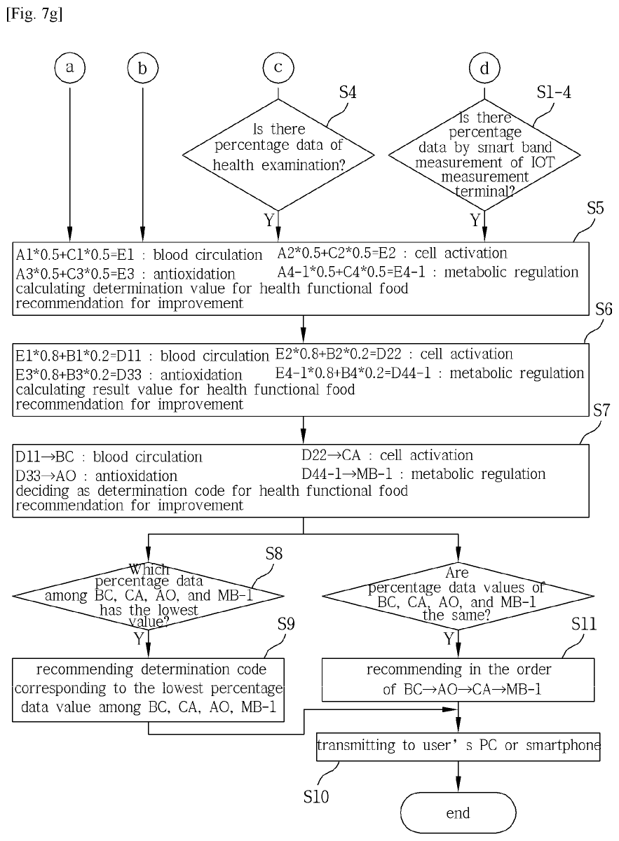

As illustrated in FIG. 7 and FIG. 7a, the method comprises the following steps: step 1 (S1) wherein if there is percentage data of health examination (blood) analysis, average percentage data obtained by summing up percentage data of questionnaire items for each of blood circulation related item (A-1), cell activation related item (A-2), antioxidation related item (A-3), and metabolism related item (A-4), among self-determination percentage data obtained by checking each of questionnaire items in a health related self-determination list related to metabolism, obesity, and hair, etc., and converting into percentages using the Likert scale of measurement, is calculated as health functional food recommendation self-phenomenon values (A1), (A2), (A3), and (A4), at a determination analysis processing unit (410A) of an analysis server (410); step 2 (S2) wherein average percentage data obtained by summing up percentage data on genetic testing items for each of blood circulation related item (B-1), cell activation related item (B-2), antioxidation related item (B-3), and metabolism related item (B-4), among genetic data input from a genetic analysis database (10), is calculated as health functional food recommendation genetic determination values (B1), (B2), (B3), and (B4) at the determination analysis processing unit (410A) of the analysis server (410); step 3 (S3) wherein percentage data obtained by summing up health examination item percentages for each of blood circulation related item (C-1), cell activation related item (C-2), antioxidation related item (C-3), and metabolism related item (C-4), which correspond to a health examination list including immunity inflammation, cardiovascular function, hyperlipidemia, thyroid, liver function, kidney, hematologic abnormality, and diabetes, etc., received from a health examination (blood) analysis company, is calculated as health functional food recommendation health examination determination values (C1), (C2), (C3), and (C4) at the determination analysis processing unit (410A) of the analysis server (410); step 4 (S4) of deciding whether there is percentage data of health examination; step 5 (S5) wherein if it is decided in step 4 (S4) that there is percentage data of health examination, self-phenomenon health functional food recommendation determination values (E1), (E2), (E3), and (E4), which are the sums of percentage data obtained by giving a recommendation weighting ratio 50%:50% to the health functional food recommendation self-phenomenon values (A1), (A2), (A3), and (A4) and the health functional food recommendation health examination determination values (C1), (C2), (C3), and (C4), are calculated at the determination analysis processing unit (410A) of the analysis server (410); step 6 (S6) wherein a recommendation weighting ratio 80%:20% is given to the self-phenomenon health functional food recommendation determination values (E1), (E2), (E3), and (E4) and the health functional food recommendation genetic determination values (B1), (B2), (B3), and (B4), and the self-phenomenon health functional food recommendation determination values (E1), (E2), (E3), and (E4) and the health functional food recommendation genetic determination values (B1), (B2), (B3), and (B4), to which the recommendation weighting ratio 80%:20% is applied, are summed up, to calculate health functional food recommendation result values (D11), (D22), (D33), and (D44) at the determination analysis processing unit (410A) of the analysis server (410); step 7 (S7) wherein the health functional food recommendation result values (D11), (D22), (D33), and (D44) calculated in step 6 (S6) are decided as health functional food recommendation determination codes (BC), (CA), (AO), and (MB), respectively, for improvement of blood circulation, cell activation, antioxidation, and metabolic regulation, at the determination analysis processing unit (410A) of the analysis server (410); step 8 (S8) of deciding which health functional food recommendation determination code among the decided health functional food recommendation determination codes (BC), (CA), (AO), and (MB) is calculated as the lowest percentage data; step 9 (S9) wherein the health functional food recommendation determination code having the lowest percentage data decided in step 8 (S8) is recommended as a health functional food recommendation determination code (BC), (CA), (AO), or (MB) at the determination analysis processing unit (410A) of the analysis server (410); and step 10 (S10) wherein the health functional food recommendation determination code recommended in step 9 (S9) is transmitted to user's PC (100) and smartphone (200) to recommend health functional food corresponding thereto to a client, and through step 11 (S11) wherein if it is decided in step 8 (S8) that the percentage data values of the health functional food recommendation result values (D11), (D22), (D33), and (D44) are the same, a determination code is decided in the order of the health food recommendation result value (D11) for blood circulation improvement→the health food recommendation result value (D33) for antioxidation improvement→the health food recommendation result value (D22) for cell activation improvement→the health food recommendation result value (D44) for metabolic regulation improvement, at the determination analysis processing unit (410A) of the analysis server (410), preferentially providing through step 10 (S10) the health functional food recommendation determination code (BC) for blood circulation improvement recommended as a priority, to provide personalized health functional food which helps health disorder that the client currently feels from self-phenomenon, and as to a health functional food recommendation determination code (OB) for obesity improvement decided by calculating a health functional food recommendation result value (D55) for obesity improvement, which will be described below, through step 8 (S8) of deciding which health functional food recommendation determination code among the decided health functional food recommendation determination codes (BC), (CA), (AO), (MB), and (OB) is calculated as the lowest percentage data, recommending health functional food corresponding thereto.

In step 5 (S5), preferably, the recommendation weighting ratio given to the health functional food recommendation self-phenomenon values (A1), (A2), (A3), and (A4) and the health functional food recommendation health examination determination values (C1), (C2), (C3), and (C4) is 50%:50% of the same percentage. The recommendation weighting ratio of the same percentage is given because health disorder that the client feels is represented by the result value obtained from blood testing, and the weighting ratio of the same percentage applied is given to self-phenomenon, thereby providing personalized health functional food suitable for the client.

Also, the aforementioned recommendation weighting ratio is provided to raise the probability of personalized food recommendation suitable for the client's health. The recommendation weighting ratio given to each of the health functional food recommendation self-phenomenon values (A1), (A2), (A3), and (A4) is greater than the recommendation weighting ratio given to each of the health functional food recommendation genetic determination values (B1), (B2), (B3), and (B4), and thus appropriate health functional food may be recommended according to self-phenomenon. Preferably, the recommendation weighting ratio 80%:20% is applied to the health functional food recommendation self-phenomenon values (A1), (A2), (A3), and (A4) and the health functional food recommendation genetic determination values (B1), (B2), (B3), and (B4).

The recommendation weighting ratio of the health functional food recommendation self-phenomenon values (A1), (A2), (A3), and (A4) and the health functional food recommendation genetic determination values (B1), (B2), (B3), and (B4) is set to 80%: 20%, because current health states may be different from inborn genetic characteristics. It is preferable to recommend health functional food in accordance with subjective phenomena which the client currently feels health disorder, and thus preferably, the greater weighting ratio is given to the self-phenomenon values by self-determination. The recommendation weighting ratio is set to 60% — 90%:40% — 10%, rather than to 60%:40%, 70%:30%, or 90%:10%. Preferably, the recommendation weighting ratio of 80%:20% is given to recommend health functional food, thereby providing the most suitable health functional food so as to solve health disorder the client currently feels. Moreover, the lower recommendation weighting ratio is given to the genetic values in order to help to solve health disorder caused by genetic factors as well, because inborn genetic characteristics cannot be ignored. Thus, the lower weighting ratio than that of the self-phenomenon values is given.

Specifically, as to the given weighting ratio, the health functional food recommendation result value (D1) for blood circulation improvement is calculated by (A1)*0.8+(B1)*0.2=(D1), which is processed at the determination analysis processing unit (410A) of the analysis server (410). The health food recommendation result value (D2) for cell activation improvement is calculated by (A2)*0.8+(B2)*0.2=(D2), the health food recommendation result value (D3) for antioxidation improvement is calculated by (A3)*0.8+(B3)*0.2=(D3), and the health food recommendation result value (D4) for metabolic regulation improvement is calculated by (A4)*0.8+(B4)*0.2=(D4), which are all processed at the determination analysis processing unit (410A) of the analysis server (410).

The health food recommendation result value (any one of D1, D2, D3, and D4) having the lowest percentage data among them is decided as a health functional food recommendation determination code (any one of BC, CA, AO, and MB) to scientifically recommend client's personalized health functional food and help to promote the client's health. For example, health functional food including vitamin C, propolis, etc., may be recommended as health functional food for antioxidation improvement; health functional food including Omega 3, CoQ10, etc., may be recommended as health functional food for blood circulation improvement; health functional food including vitamin D may be recommended as health functional food for metabolic regulation improvement; and health functional food including magnesium may be recommended as health functional food for cell activation improvement.

b) Health functional food recommendation for obesity improvement

As shown in FIG. 7 and FIG. 7b, an obesity related health functional food recommendation self-phenomenon value (A5) is calculated in step 1 (S1), a health functional food recommendation genetic determination value (B5) is calculated in step 2 (S2), and a health functional food recommendation health examination determination value (C5) is calculated in step 3 (S3). Then, through step 4-1 (S4-1) of deciding whether there is percentage data of health examination; step 5-1 (S5-1) wherein if it is decided in step 4-1 (S4-1) that there is percentage data of health examination, a self-phenomenon health functional food recommendation determination value (E5), which is the sum of percentage data obtained by giving a recommendation weighting ratio 50%:50% to the health functional food recommendation self-phenomenon value (A5) and the health functional food recommendation health examination determination value (C5), is calculated at the determination analysis processing unit (410A) of the analysis server (410); step 6-1 (S6-1) wherein a recommendation weighting ratio 80%:20% is given to the self-phenomenon health functional food recommendation determination value (E5) and the health functional food recommendation genetic determination value (B5), and the self-phenomenon health functional food recommendation determination value (E5) and the health functional food recommendation genetic determination value (B5), to which the recommendation weighting ratio 80%:20% is applied, are summed up, to calculate a health functional food recommendation result value (D55) at the determination analysis processing unit (410A) of the analysis server (410); step 12 (S12) of deciding whether the rate of the percentage data value of the health functional food recommendation result value (D55) calculated in step 6-1 (S6-1) is 45% or less; step 13 (S13) wherein if it is decided in step 12 (S12) that the rate of the percentage data value of the health functional food recommendation result value (D55) is 45% or less, the result value is decided as a health functional food recommendation determination code (OB) for obesity regulation improvement; and step 10-1 (S10-1) wherein the health functional food recommendation determination code recommended in step 13 (S13) is transmitted to user's PC (100) and smartphone (200) to recommend health functional food for obesity regulation improvement to a client, health functional food may be recommended to the client according to the health functional food recommendation result value (D55) for obesity regulation improvement to take health functional food for obesity improvement. As health functional food for obesity regulation, food including Catechin, Banaba, etc., may be included.

B. Hereinafter, in case there is no percentage data of health examination (blood) analysis, a method for recommending health functional food for improvement of metabolism and hair loss by a comparison between health functional food recommendation self-phenomenon values and health functional food genetic determination values, is described.

a) Method for recommending health functional food for metabolism improvement

As shown in FIG. 7 and FIG. 7c, if there is no percentage data of health examination (blood) analysis, health functional food recommendation self-phenomenon values (A1), (A2), (A3), and (A4) are calculated in step 1 (S1), and health functional food recommendation genetic determination values (B1), (B2), (B3), and (B4) are calculated in step 2 (S2). Then, through step 6-2 (S6-2) wherein a health functional food recommendation weighting ratio 80%:20% is given to the health functional food recommendation self-determination values (A1), (A2), (A3), and (A4) and the health functional food recommendation genetic determination values (B1), (B2), (B3), and (B4), and the self-phenomenon health functional food recommendation determination values (E1), (E2), (E3), and (E4) and the health functional food recommendation genetic determination values (B1), (B2), (B3), and (B4), to which the recommendation weighting ratio 80%:20% is applied, are summed up, to calculate health functional food recommendation result values (D1), (D2), (D3), and (D4) at the determination analysis processing unit (410A) of the analysis server (410); step 7-1 (S7-1) wherein the health functional food recommendation result values (D1), (D2), (D3), and (D4) calculated in step 6-2 (S6-2) are decided as health functional food recommendation determination codes (BC-1), (CA-1), (AO-1), and (MB-1), respectively, for improvement of blood circulation, cell activation, antioxidation, and metabolic regulation, at the determination analysis processing unit (410A) of the analysis server (410); step 8-1 (S8-1) of deciding which health functional food recommendation determination code among the decided health functional food recommendation determination codes (BC-1), (CA-1), (AO-1), and (MB-1) is calculated as the lowest percentage data; step 9-1 (S9-1) wherein the health functional food recommendation determination code having the lowest percentage data decided in step 8-1 (S8-1) is recommended as a health functional food recommendation determination code (BC-1), (CA-1), (AO-1), or (MB-1) at the determination analysis processing unit (410A) of the analysis server (410); and step 10-2 (S10-2) wherein the health functional food recommendation determination code recommended in step 9-1 (S9-1) is transmitted to user's PC (100) and smartphone (200) to recommend health functional food corresponding thereto to a client, and through step 11-1 (S11-1) wherein if it is decided in step 8-1 (S8-1) that the percentage data values of the health functional food recommendation result values (D1), (D2), (D3), and (D4) are the same, a determination code is decided in the order of the health food recommendation result value (D1) for blood circulation improvement→the health food recommendation result value (D3) for antioxidation improvement→the health food recommendation result value (D2) for cell activation improvement→the health food recommendation result value (D4) for metabolic regulation improvement, at the determination analysis processing unit (410A) of the analysis server (410), a health functional food recommendation determination code (BC-1) for blood circulation improvement recommended as a priority is preferentially provided through step 10-2 (S10-2), to provide personalized health functional food which helps health disorder that the client currently feels from self-phenomenon.

b) Hair loss improvement by a comparison between self-phenomenon value and genetic value

As shown in FIG. 7 and FIG. 7d, a hair loss related health functional food recommendation self-phenomenon value (A6) and a hair loss related health functional food recommendation genetic determination value (B6) are calculated in step 1 (S1) and step 2 (S2). Then, through step 6-3 (S6-3) wherein a health functional food recommendation result value (D66) is calculated, which is the sum of percentage data obtained by applying a recommendation weighting ratio 80%:20% to the health functional food recommendation self-phenomenon value (A6) and the health functional food recommendation genetic determination value (B6); step 12-1 (S12-1) of deciding whether the rate of the percentage data value calculated in step 6-3 (S6-3) is 45% or less; step 13-1 (S13-1) wherein if it is decided in step 12-1 (S12-1) that the rate of the percentage data value is 45% or less, the result value is decided as a health functional food recommendation determination code (HL) for hair loss improvement; and step 10-3 (S10-3) wherein the health functional food recommendation determination code recommended in step 13-1 (S13-1) is transmitted to user's PC (100) and smartphone (200) to recommend health functional food for hair loss improvement to a client, health functional food for hair loss improvement is provided. As health functional food for hair loss improvement, food including BioTin, etc., for scalp reinforcement may be included.

c) Method for recommending health functional food for obesity improvement according to body mass index (BMI) measurement

As shown in FIG. 7 and FIG. 7e, a body mass index related health functional food recommendation self-phenomenon value (A7) and a body mass index related health functional food recommendation genetic determination value (B7) are calculated in step 1 (S1) and step 2 (S2). Then, through step 6-4 (S6-4) wherein a health functional food recommendation result value (D77) is calculated, which is the sum of percentage data obtained by applying a recommendation weighting ratio 80%:20% to the health functional food recommendation self-phenomenon value (A7) and the health functional food recommendation genetic determination value (B7); step 12-2 (S12-2) of deciding whether the rate of the percentage data value calculated in step 6-4 (S6-4) is 45% or less; step 13-2 (S13-2) wherein if it is decided in step 12-2 (S12-2) that the rate of the percentage data value is 45% or less, the result value is decided as a health functional food recommendation determination code (BM) for body mass index improvement; and step 10-4 (S10-4) wherein the health functional food recommendation determination code recommended in step 13-2 (S13-2) is transmitted to user's PC (100) and smartphone (200) to recommend health functional food for body mass index improvement to a client, health functional food for body mass index improvement is provided. The method of calculating the body mass index related health functional food recommendation self-phenomenon value (A7) at the determination analysis processing unit (410A) of the analysis server (410) is typically performed by automatically calculating weight (kg)÷(height (m)×height (m)) once the client fills in his weight and height in the self-determination list of questionnaire items. According to the BMI standards of the Korean Society for the Study of Obesity, if the BMI is 15˜18.4 kg/m², it is classified as underweight and calculated into a phenomenon section value 46˜18.5%; if the BMI is 18.5˜22.9 kg/m², it is classified as normal and calculated into a phenomenon section value 61˜85%; if the BMI is 23˜24.9 kg/m², it is classified as overweight and calculated into a phenomenon section value 46˜60%; if the BMI is 25˜29.9 kg/m², it is classified as obese and calculated into a phenomenon section value 31˜45%; and if the BMI is 30˜35 kg/m², it is classified as severe obese and calculated into a phenomenon section value 15˜30%. If the health functional food recommendation result value (D77) obtained by applying a recommendation weighting ratio 80%:20% to the health functional food recommendation self-phenomenon value (A7), which is the calculated phenomenon section value, and the health functional food recommendation genetic determination value (B7), is 45% or less, the result value is decided as a health functional food recommendation determination code (BM) for obesity improvement according to the body mass index.

C. Method for recommending health functional food based on percentage data of hair loss related items obtained by the scalp measurement device (510) of the IoT measurement terminal (500), the percentage data information of body mass index (BMI) obtained by the weighing scale (520) and smart band (530), and the percentage data of metabolic regulation obtained from blood pressure measurement

In case there are percentage data of hair loss related items obtained by the scalp measurement device (510) of the IoT measurement terminal (500), percentage data information of body mass index (BMI) obtained by the weighing scale (520) and smart band (530), and percentage data of metabolic regulation obtained from blood pressure measurement,

a) as shown in FIG. 7 and FIG. 7d, a health functional food recommendation self-phenomenon value (A6-1) is calculated based on the percentage data of hair loss related items by the scalp measurement device (510) of the IoT measurement terminal (500), and a hair loss related health functional food recommendation genetic determination value (B6) is calculated in step 2 (S2). Then, through step 1-2 (S1-2) of deciding whether there is percentage data of hair loss related items by the scalp measurement device (510) of the IoT measurement terminal (500); step 6-5 (S6-5) wherein if it is decided in step 1-2 (S1-2) that there is percentage data of hair loss related items by the scalp measurement device (510) of the IoT measurement terminal (500), a health functional food recommendation result value (D66-1) is calculated, which is the sum of percentage data obtained by applying a recommendation weighting ratio 80%:20% to the health functional food recommendation self-phenomenon value (A6-1) and the health functional food recommendation genetic determination value (B6); step 12-3 (S12-3) of deciding whether the rate of the percentage data value calculated in step 6-5 (S6-5) is 45% or less; step 13-3 (S13-3) wherein if it is decided in step 12-3 (S12-3) that the rate of the percentage data value is 45% or less, the result value is decided as a health functional food recommendation determination code (HL-1) for hair loss improvement; and step 10-3 (S10-3) wherein the health functional food recommendation determination code (HL-1) recommended in step 13-3 (S13-3) is transmitted to user's PC (100) and smartphone (200) to recommend health functional food for hair loss improvement to a client, health functional food for hair loss improvement may be provided.

b) As shown in FIG. 7 and FIG. 7f, a body mass index related health functional food recommendation self-phenomenon value (A7) and a body mass index related health functional food recommendation genetic determination value (B7) are calculated in step 1 (S1) and step 2 (S2). Then, through step 6-4 (S6-4) wherein a health functional food recommendation result value (D77) is calculated, which is the sum of percentage data obtained by applying a recommendation weighting ratio 80%:20% to the health functional food recommendation self-phenomenon value (A7) and the health functional food recommendation genetic determination value (B7); step 12-2 (S12) of deciding whether the rate of the percentage data value calculated in step 6-4 (S6-4) is 45% or less; step 13-2 (S13-2) wherein if it is decided in step 12-2 (S12-2) that the rate of the percentage data value is 45% or less, the result value is decided as a health functional food recommendation determination code (BM) for body mass index improvement; and step 10-3 (S10-3) wherein the health functional food recommendation determination code recommended in step 13-2 (S13-2) is transmitted to user's PC (100) and smartphone (200), health functional food related to body mass index may be recommended. Or, if percentage data of body mass index (BMI) obtained by the weighing scale (520) of the IoT measurement terminal (500) is calculated, a health functional food recommendation self-phenomenon value (A7-1) based on the percentage data of body mass index (BMI) obtained by the weighing scale (520) of the IoT measurement terminal (500), instead of the health functional food recommendation self-phenomenon value (A7), is calculated in step 1-1 (S1-1), and a body mass index related health functional food recommendation genetic determination value (B7) is calculated in step 2 (S2). Then, through step 1-3 (S1-3) of deciding whether there is percentage data of body mass index obtained by the weighing scale (520) of the IoT measurement terminal (500); step 6-6 (S6-6) wherein if it is decided in step 1-3 (S1-3) that there is percentage data of body mass index obtained by the weighing scale (520) of the IoT measurement terminal (500), a health functional food recommendation result value (D77) is calculated, which is the sum of percentage data obtained by applying a recommendation weighting ratio 80%:20% to the health functional food recommendation self-phenomenon value (A7-1) and the health functional food recommendation genetic determination value (B7); step 12-4 (S12-4) of deciding whether the rate of the percentage data value calculated in step 6-6 (S6-6) is 45% or less; step 13-4 (S13-4) wherein if it is decided in step 12-4 (S12-4) that the rate of the percentage data value is 45% or less, the result value is decided as a health functional food recommendation determination code (BM-1) for body mass index improvement; and step 10-3 (S10-3) wherein the health functional food recommendation determination code (BM-1) recommended in step 13-4 (S13-4) is transmitted to user's PC (100) and smartphone (200) to recommend health functional food for hair loss improvement to a client, health functional food for body mass index improvement may be provided.

c) As shown in FIG. 7 and FIG. 7g, health functional food recommendation self-phenomenon values (A1), (A2), and (A3) are calculated in step 1 (S1), a metabolism related health functional food recommendation self-phenomenon value (A4-1) by the smart band (530) of the IoT measurement terminal (500) is calculated in step 1-1 (S1-1); step 1-4 (S1-4) of deciding whether there is metabolism related percentage data by the smart band (530) of the IoT measurement terminal (500) in step 1-1 (S1-1); health functional food recommendation genetic determination values (B1), (B2), (B3), and (B4) are calculated in step 2 (S2); and health functional food recommendation health examination determination values (C1), (C2), (C3), and (C4) are calculated in step 3 (S3). By comprising step 4 (S4) of deciding whether there is percentage data of health examination; step 5 (S5) wherein if it is decided in step 4 (S4) that there is percentage data of health examination, self-phenomenon health functional food recommendation determination values (E1), (E2), (E3), and (E4-1), which are the sums of percentage data obtained by giving a recommendation weighting ratio 50%:50% to the health functional food recommendation self-phenomenon values (A1), (A2), (A3), and (A4-1) and the health functional food recommendation health examination determination values (C1), (C2), (C3), and (C4), are calculated at the determination analysis processing unit (410A) of the analysis server (410); step 6 (S6) wherein a recommendation weighting ratio 80%:20% is given to the self-phenomenon health functional food recommendation determination values (E1), (E2), (E3), and (E4-1) and the health functional food recommendation genetic determination values (B1), (B2), (B3), and (B4), and the self-phenomenon health functional food recommendation determination values (E1), (E2), (E3), and (E4-1) and the health functional food recommendation genetic determination values (B1), (B2), (B3), and (B4), to which the recommendation weighting ratio 80%:20% is applied, are summed up, to calculate health functional food recommendation result values (D11), (D22), (D33), and (D44-1) at the determination analysis processing unit (410A) of the analysis server (410); step 7 (S7) wherein the health functional food recommendation result values (D11), (D22), (D33), and (D44-1) calculated in step 6 (S6) are decided as health functional food recommendation determination codes (BC), (CA), (AO), and (MB-1), respectively, for improvement of blood circulation, cell activation, antioxidation, and metabolic regulation, at the determination analysis processing unit (410A) of the analysis server (410); step 8 (S8) of deciding which health functional food recommendation determination code among the decided health functional food recommendation determination codes (BC), (CA), (AO), and (MB-1) is calculated as the lowest percentage data; step 9 (S9) wherein the health functional food recommendation determination code having the lowest percentage data decided in step 8 (S8) is recommended as a health functional food recommendation determination code (BC), (CA), (AO), or (MB-1) at the determination analysis processing unit (410A) of the analysis server (410); and step 10 (S10) wherein the health functional food recommendation recommended determination code recommended in step 9 (S9) is transmitted to user's PC (100) and smartphone (200) to recommend health functional food corresponding thereto to a client; and through step 11 (S11) wherein if it is decided in step 8 (S8) that the percentage data values of the health functional food recommendation result values (D11), (D22), (D33), and (D44-1) are the same, a determination code is decided in the order of the health food recommendation result value (D11) for blood circulation improvement→the health food recommendation result value (D33) for antioxidation improvement→the health food recommendation result value (D22) for cell activation improvement→the health food recommendation result value (D44-1) for metabolic regulation improvement, at the determination analysis processing unit (410A) of the analysis server (410), a health functional food recommendation determination code (BC) for blood circulation improvement recommended as a priority is preferentially provided through step 10 (S10), to provide personalized health functional food which helps health disorder that the client currently feels from self-phenomenon.

The present invention is explained in detail as above, and the explanation is not intended to limit the scope of the present invention. The explanation is provided for illustrative purposes only, and the scope of the present invention shall be defined by the accompanying claims. It should be construed that simple modifications or alternations within the scope of the claims fall within the scope of the present invention.

What is claimed is:

1. A system for providing personalized health functional food information, comprising:
a genetic analysis database (10) for analyzing percentage data on health related genetic testing items related to metabolism, obesity, and hair genes, received from a genetic analysis company;
a self-determination information input unit (300) for inputting, through Internet (I) or Wi-Fi (W1), self-determination percentage data obtained by checking, at a user's PC (100) and smartphone (200), each of questionnaire items of metabolism, obesity, and hair (hair loss), etc. in a health related self-determination list and converting into percentages using the Likert scale of measurement;
a data storage unit (410B) for storing the genetic percentage data of the genetic analysis database (10) and the self-determination percentage data from the self-determination information input unit (300) and a determination analysis processing unit (410A) for comparing and analyzing input data, which constitute an analysis server (410) in a cloud server (400); and
a health examination (blood) analysis database (420) for analyzing and storing percentage data on health examination items received from a health examination (blood) analysis company and transmitting the data to the determination analysis processing unit (410A); and an IoT measurement data input unit (500A) for inputting, through Bluetooth (BT) or Wi-Fi (W2), percentage data of hair loss related items obtained by a scalp measurement device (510) of an IoT measurement terminal (500), percentage data of body mass index (BMI) obtained by a weighing scale (520) and a smart band (530), and percentage data of metabolic regulation obtained from blood pressure measurement,
wherein if there is data input by the IoT measurement data input unit (500A), the percentage data of body mass index obtained by the weighing scale (520) and smart band (530) and the percentage data of metabolic regulation obtained from blood pressure measurement, instead of the corresponding items of the self-determination percentage data obtained by checking each of the questionnaire items in the self-determination list and converting into percentages using the Likert scale of measurement, are input to the analysis server (410) for analysis, the percentage data of hair loss related items obtained by the scalp measurement device (510) is input to an image analysis server (430) which comprises a reference database (430A) and a scalp measurement data analysis unit (430B) and analyzed by the analysis server (410) to analyze client's genetic percentage data, client's body related information data by self-determination, and health examination information data, self-phenomenon health functional food recommendation determination values (E1), (E2), (E3), and (E4), which are calculated by giving a health functional food recommendation percentage weighting ratio 50%:50% to metabolism related health functional food recommendation self-phenomenon values (A1), (A2), (A3), and (A4), calculated as average percentage data from the questionnaire items in the self-determination list, and metabolism related health functional food recommendation health examination determination values (C1), (C2), (C3), and (C4), calculated as average percentage data from health examination, and summing up the values, are calculated, a recommendation weighting ratio 80%:20% is given to each of the self-phenomenon health functional food recommendation determination values (E1), (E2), (E3), and (E4) and health functional food recommendation genetic determination values (B1), (B2), (B3), and (B4), the self-phenomenon health functional food recommendation determination values (E1), (E2), (E3), and (E4) and the health functional food recommendation genetic determination values (B1), (B2), (B3), and (B4), to which the recommendation weighting ratio 80%:20% is applied, are summed up, to calculate health functional food recommendation result values (D11), (D22), (D33), and (D44), the calculated health functional food recommendation result values (D11), (D22), (D33), and (D44) are decided as health functional food recommendation determination codes (BC), (CA), (AO), and (MB), respectively, for improvement of blood circulation, cell activation, antioxidation, and metabolic regulation, at the determination analysis processing unit (410A) of the analysis server (410), and a health functional food recommendation determination code of the metabolism item, calculated as the health functional food recommendation result value having the lowest percentage data among the decided health functional food recommendation determination codes (BC), (CA), (AO), and (MB), is decided for health functional food, or if the percentage data values of the health functional food recommendation result values are the same, a determination code is decided in the order of the health food recommendation result value for blood circulation improvement→the health food recommendation result value for antioxidation improvement→the health food recommendation result value for cell activation improvement→the health food recommendation result value for metabolic regulation improvement, at the determination analysis processing unit (410A) of the analysis server (410), to transmit it to the user's PC (100) and smartphone (200).

2. The system of claim 1 of, if the percentage data of each of the health functional food recommendation result values for improvement of obesity, body mass index, and hair loss calculated by giving a health functional food recommendation percentage weighting ratio 80%:20% to each of the health functional food recommendation self-phenomenon values related to obesity, body mass index, and hair loss obtained by checking each of the questionnaire items in the health related self-determination list and converting into percentages using the Likert scale of measurement and the health functional food genetic determination values related to obesity, body mass index, and hair loss, is 45% or less, recommending personalized health functional food corresponding thereto for improvement of obesity and hair loss.

3. A method for providing personalized health functional food using the system for providing personalized health functional food of claim 1, comprising:
step 1 (S1) wherein if there is percentage data of health examination (blood) analysis, average percentage data obtained by summing up percentage data of questionnaire items for each of blood circulation related item (A-1), cell activation related item (A-2), antioxidation related item (A-3), and metabolism related item (A-4), obesity related item (A-5), hair loss related item (A-6), and body mass index related item (A-7) among self-determination percentage data obtained by checking each of questionnaire items in a health related self-determination list related to metabolism, obesity, and hair, etc., and converting into percentages using the Likert scale of measurement, is calculated as health functional food recommendation self-phenomenon values (A1), (A2), (A3), (A4), (A5), (A6), and (A7), at a determination analysis processing unit (410A) of an analysis server (410);
step 2 (S2) wherein average percentage data obtained by summing up percentage data on genetic testing items for each of blood circulation related item (B-1), cell activation related item (B-2), antioxidation related item (B-3), metabolism related item (B-4), obesity related item (B-5), hair loss related item (B-6), and body mass index related item (B-7) among genetic data input from a genetic analysis database (10), is calculated as health functional food recommendation genetic determination values (B1), (B2), (B3), (B4), (B5), (B6), and (B7) at the determination analysis processing unit (410A) of the analysis server (410);
step 3 (S3) wherein percentage data obtained by summing up health examination item percentages for each of blood circulation related item (C-1), cell activation related item (C-2), antioxidation related item (C-3), metabolism related item (C-4), and obesity related item (C-5), which correspond to a health examination list including immunity inflammation, cardiovascular function, hyperlipidemia, thyroid, liver function, kidney, hematologic abnormality, and diabetes, etc., received from a health examination (blood) analysis company, is calculated as health functional food recommendation health examination determination values (C1), (C2), (C3), (C4), and (C5) at the determination analysis processing unit (410A) of the analysis server (410);
step 4 (S4) of deciding whether there is percentage data of health examination;
step 5 (S5) wherein if it is decided in step 4 (S4) that there is percentage data of health examination, self-phenomenon health functional food recommendation determination values (E1), (E2), (E3), and (E4), which are the sums of percentage data obtained by giving a recommendation weighting ratio 50%:50% to the health functional food recommendation self-phenomenon values (A1), (A2), (A3), and (A4) and the health functional food recommendation health examination determination values (C1), (C2), (C3), and (C4), are calculated at the determination analysis processing unit (410A) of the analysis server (410);
step 6 (S6) wherein a recommendation weighting ratio 80%:20% is given to the self-phenomenon health functional food recommendation determination values (E1), (E2), (E3), and (E4) and the health functional food recommendation genetic determination values (B1), (B2), (B3), and (B4), and the self-phenomenon health functional food recommendation determination values (E1), (E2), (E3), and (E4) and the health functional food recommendation genetic determination values (B1), (B2), (B3), and (B4), to which the recommendation weighting ratio 80%:20% is applied, are summed up, to calculate health functional food recommendation result values (D11), (D22), (D33), and (D44) at the determination analysis processing unit (410A) of the analysis server (410);
step 7 (S7) wherein the health functional food recommendation result values (D11), (D22), (D33), and (D44) calculated in step 6 (S6) are decided as health functional food recommendation determination codes (BC), (CA), (AO), and (MB), respectively, for improvement of blood circulation, cell activation, antioxidation, and metabolic regulation, at the determination analysis processing unit (410A) of the analysis server (410);
step 8 (S8) of deciding which health functional food recommendation determination code among the decided health functional food recommendation determination codes (BC), (CA), (AO), and (MB) is calculated as the lowest percentage data;
step 9 (S9) wherein the health functional food recommendation determination code having the lowest percentage data decided in step 8 (S8) is recommended as a health functional food recommendation determination code (BC), (CA), (AO), or (MB) at the determination analysis processing unit (410A) of the analysis server (410); and
step 10 (S10) wherein the health functional food recommendation determination code recommended in step 9 (S9) is transmitted to a user's PC (100) and smartphone (200) to recommend health functional food corresponding thereto to a client, and through step 11 (S11),
wherein if it is decided in step 8 (S8) that the percentage data values of the health functional food recommendation result values (D11), (D22), (D33), and (D44) are the same, a determination code is decided in the order of the health food recommendation result value (D11) for blood circulation improvement→the health food recommendation result value (D33) for antioxidation improvement→the health food recommendation result value (D22) for cell activation improvement→the health food recommendation result value (D44) for metabolic regulation improvement, at the determination analysis processing unit (410A) of the analysis server (410), preferentially providing through step 10 the health functional food recommendation determination code (BC) for blood circulation improvement recommended as a priority (S10), to provide personalized health functional food which helps health disorder that a client currently feels from self-phenomenon.

4. The method of claim 3, wherein an obesity related health functional food recommendation self-phenomenon value (A5) is calculated in step 1 (S1), a health functional food recommendation genetic determination value (B5) is calculated in step 2 (S2), and a health functional food recommendation health examination determination value (C5) is calculated in step 3 (S3), and then, through step 4-1 (S4-1) of deciding whether there is percentage data of health examination; step 5-1 (S5-1) wherein if it is decided in step 4-1 (S4-1) that there is percentage data of health examination, a self-phenomenon health functional food recommendation determination value (E5), which is the sum of percentage data obtained by giving a recommendation weighting ratio 50%:50% to the health functional food recommendation self-phenomenon value (A5) and the health functional food recommendation health examination determination value (C5), is calculated at the determination analysis processing unit (410A) of the analysis server (410); step 6-1 (S6-1) wherein a recommendation weighting ratio 80%:20% is given to the self-phenomenon health functional food recommendation determination value (E5) and the health functional food recommendation genetic determination value (B5), and the self-phenomenon health functional food recommendation determination value (E5) and the health functional food recommendation genetic determination value (B5), to which the recommendation weighting ratio 80%:20% is applied, are summed up, to calculate a health functional food recommendation result value (D55) at the determination analysis processing unit (410A) of the analysis server (410); step 12 (S12) of deciding whether the rate of the percentage data value of the health functional food recommendation result value (D55) calculated in step 6-1 (S6-1) is 45% or less; step 13 (S13) wherein if it is decided in step 12 (S12) that the rate of the percentage data value of the health functional food recommendation result value (D55) is 45% or less, the result value is decided as a health functional food recommendation determination code (OB) for obesity regulation improvement; and step 10-1 (S10-1) wherein the health functional food recommendation determination code recommended in step 13 (S13) is transmitted to the user's PC (100) and smartphone (200) to recommend health functional food for obesity regulation improvement to a client, health functional food is recommended to the client according to the health functional food recommendation result value (D55) for obesity regulation improvement to provide health functional food for obesity improvement.

5. The method of claim 3, wherein if there are health functional food recommendation self-phenomenon values (A4-1), (A6-1), and (A7-1) related to metabolism, body mass index, and hair loss, respectively, from percentage data of hair loss related items obtained by a scalp measurement device (510) of an IoT measurement terminal (500) and measurement by a weighing scale (520) and a smart band (530), among the health functional food recommendation self-phenomenon values (A1), (A2), (A3), (A4), (A5), (A6), and (A7) calculated in step 1 (S1), the health functional food recommendation self-phenomenon values (A4-1), (A6-1), and (A7-1) are substituted for the health functional food recommendation self-phenomenon values (A4), (A6), and (A7), to recommend health functional food for improvement of metabolism, hair loss, and obesity.
